(19) United States
(12) Patent Application Publication
(10) Pub. No.: US 2020/0276019 A1
(43) Pub. Date: Sep. 3, 2020

(54) THREE DIMENSIONALLY PRINTED AND NANOCOATED MEDICAL IMPLANTS

(71) Applicant: PrinterPrezz, Inc., Fremont, CA (US)

(72) Inventors: Shrinivas Shetty, Fremont, CA (US); Pawan Kapur, Fremont, CA (US)

(21) Appl. No.: 16/785,558

(22) Filed: Feb. 7, 2020

Related U.S. Application Data
(60) Provisional application No. 62/802,702, filed on Feb. 7, 2019.
(60) Provisional application No. 62/805,265, filed on Feb. 13, 2019.
(60) Provisional application No. 62/805,269, filed on Feb. 13, 2019.

(51) Int. Cl. A61F 2/30; A61F 2/44; A61F 2/34; A61F 2/36; A61F 2/28; B33Y 80/00; B33Y 40/20

(52) U.S. Cl. CPC A61F 2/3094; A61F 2002/30593; A61F 2/34; A61F 2/36; A61F 2/2875; B33Y 80/00; B33Y 40/20; A61F 2310/0052; A61F 2002/30677; A61F 2310/00023; A61F 2002/30985; A61F 2002/3084; A61F 2002/30838; A61F 2002/30784; A61F 2/4455

(57) ABSTRACT

Fabrication methods and structures for three dimensional medical implants are provided.

### CROSS-REFERENCE TO RELATED APPLICATIONS

This application claims priority to U.S. Provisional Patent Application Ser. Nos. 62/802702 filed Feb. 7, 2019, App. No. 62/802706 filed Feb. 7, 2019, App. No. 62/805265 filed Feb. 13, 2019, and App. No. 62/805269 filed Feb. 7, 2019 all of which are hereby incorporated by reference in their entirety.

### FIELD

The present disclosure relates in general to the fields of medical implants, and more particularly to three dimensional medical implants.

### BACKGROUND

Three dimensionally 3D printed (i.e., additively manufactured) spine implants such as interbody cages and screws as well as other medical implants are already, in some instances, advantageously more advanced than their conventional counterparts made using subtractive manufacturing. These 3D printed implants are typically small metallic or plastic structures and, by design, possess significant structural complexity in the form of lattices, porosity, and holes (e.g., see FIG. 1 showing examples of 3D printed spinal implants and FIG. 2 showing examples of 3D printed hip and face implants). Generally, 3D printed implants benefit from structural intracies which cannot be traditionally fabricated using conventional subtractive manufacturing. These 3D printed implants and the structural intricacies of these implants provide several advantages: first, the structural intricacies provide a scaffold for bone growth, increasing osseointegration; second, the structural intricacies present a much larger surface area, also resulting in an increased osseointegration; third, using porosity and intricate lattices, these implants may be made to match the physical and structural properties of the native bone, such as its Young's modulus or otherwise optimise it for healing. This, in turn, reduces stress shielding and age-induced loosening of the implant, and several studies have demonstrated advantageous modulation in osseointegration with porosity and lattice structures using these implants.

3D printing may also be more conducive to working with biocompatible materials such as titanium, PEEK, and PEKK as compared to conventional manufacturing. Titanium, while being an ideal metal for medical implants due to several highly advantageous properties, nevertheless is difficult to manipulate and mold using conventional manufacturing. Titanium's strength to weight ratio is one of the highest amongst metals; it is resistant to chemical reactions with most compounds; it is non-toxic and biocompatible; and, it is resilient to rust as it readily forms titanium oxide which serves as a highly robust and impermeable barrier to most corrosive agents. In medical applications, titanium's strength may be further fortified by alloying it with 6% aluminum and 4% vanadium, while its ductility and fracture toughness may be enhanced by reducing contaminants through extra low interstitials (ELI) grade alloys such as extra low interstitial titanium alloy Ti6Al4V.

While 3D printing may advance medical implant structures, many challenges and problems relating to these second-generation devices persist including being prone to infections. Because of a large surface area stemming from lattices and porosity, these structurally complex implants may be a fertile ground for bacteria growth as well as susceptible to other undesirable contaminants. These problems may result in revision surgeries, which, in turn, impacts the quality of life for patients, reduces patient care due to limited resources, and can dramatically drive up the cost for hospitals and patients.

### BRIEF SUMMARY OF THE INVENTION

Therefore, a need has arisen for three dimensional medical implants having structural intricacies, such as porosity and lattices, and provide improved anti-infection properties. In accordance with the disclosed subject matter, three dimensional medical additively manufactured implants are provided which substantially eliminate or reduce disadvantages and deficiencies associated with previously developed three dimensional additively manufactured medical implants.

According to one aspect of the disclosed subject matter, a method for forming a medical implant is provided. A three dimensional structure formed using three dimensional (3D) printing and having x, y, and z structural geometry in a three dimensional x, y, and z Cartesian coordinate system is formed. The three dimensional structure is coated with an anti-infection material having a thickness in the range of 0.5 nanometers to 1.0 micrometers.

According to another aspect of the disclosed subject matter, a medical implant is provided. A three dimensional structural component has x, y, and z structural geometry in a three dimensional x, y, and z Cartesian coordinate system and is made of at least a structural material and a nanocoating. The nanocoating is an anti-infection material having a thickness in the range of 0.5 nanometers to 1.0 micrometers and coats three dimensional structural material.

These and other aspects of the disclosed subject matter, as well as additional novel features, will be apparent from the description provided herein. The intent of this summary is not to be a comprehensive description of the claimed subject matter, but rather to provide a short overview of some of the subject matter's functionality. Other systems, methods, features and advantages here provided will become apparent to one with skill in the art upon examination of the following FIGUREs and detailed description. It is intended that all such additional systems, methods, features and advantages that are included within this description, be within the scope of any claims.

### BRIEF DESCRIPTION OF THE DRAWINGS

The features, natures, and advantages of the disclosed subject matter may become more apparent from the detailed description set forth below when taken in conjunction with the drawings in which like reference numerals indicate like features and wherein:

FIG. 1 is a drawing showing examples medical spine implants;

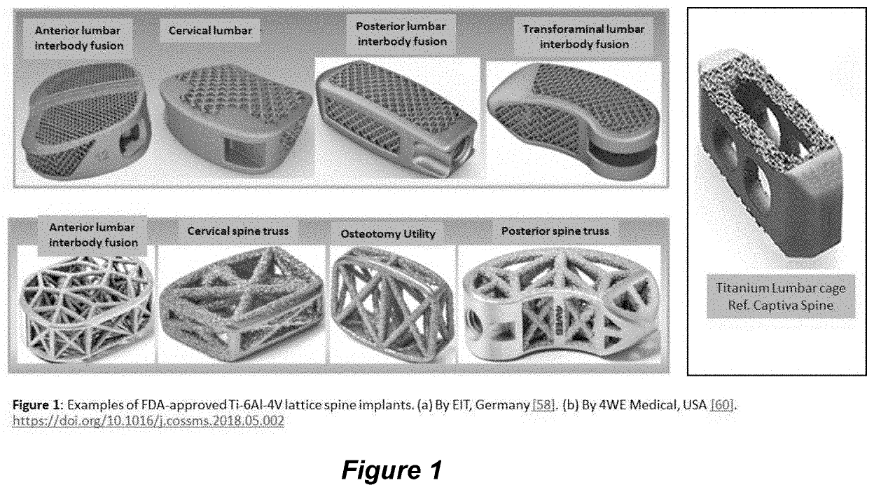

FIG. 2 is a drawing showing examples of medical hip and face implants; and

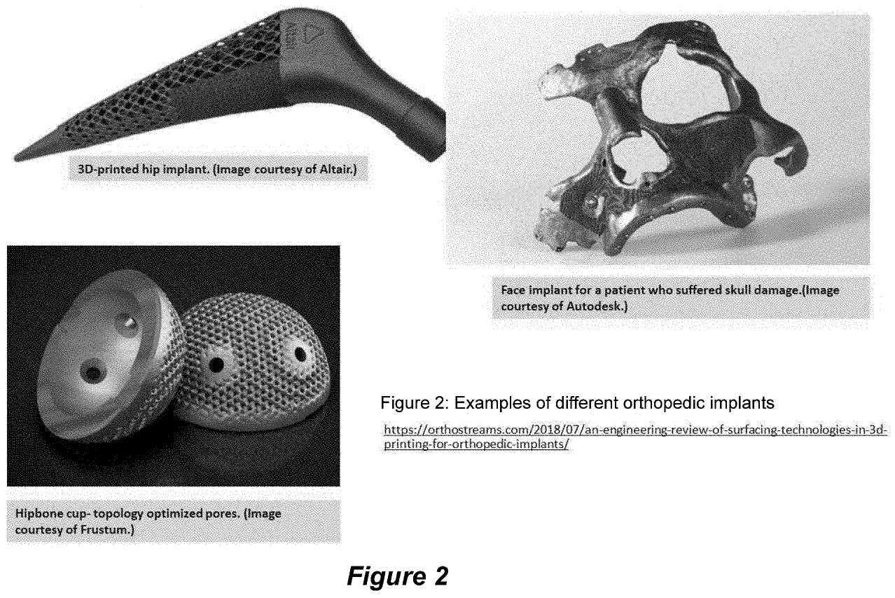

FIG. 3 is a drawing showing disclosed subject matter solutions as compared to state of the art solutions.

### DETAILED DESCRIPTION

The following description is not to be taken in a limiting sense, but is made for the purpose of describing the general principles of the present disclosure. The scope of the present disclosure should be determined with reference to the claims. Exemplary embodiments of the present disclosure are illustrated in the drawings, like numbers being used to refer to like and corresponding parts of the various drawings. The dimensions of drawings provided are not shown to scale.

As used in the description of the embodiments and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

As used herein, the term infection is used to refer to the presence of any undesirable pathogen, most commonly bacteria, but may also include prions, viruses, protozoa, fungi, nematodes, as well as abnormal human cells. Colonization is used interchangeably with infection.

As used herein, the term PEEK is used to refer both to polyether ether ketone, a colourless organic thermoplastic polymer as well as the larger polyaryletherketone (PAEK) family such as PEKK (polyetherketoneketone).

As used herein, the term “anti-infection” coating is used to refer a coating, which comprises a single material or composite of materials that provides active and/or passive effects that ultimately decrease the risk of infection. An active effect would be a bactericidal or bacteriostatic effect in which bacteria is killed or the ability for the bacteria to reproduce is impaired. Another active effect would be enhancement of the host immune system to defend against bacteria. A passive effect would be change in the surface properties that affects the the ability for a pathogen to adhere and colonize the implant.

It is understood that a composite of materials can consist of a homogeneous or heterogenous mix of multiple materials. The materials may be doped heterogeneously at different proportions (i.e. 10% silver) exposed to the surface. The coatings may be applied serially in which a copper-based coating is applied to an implant and then a graphene-based coating is applied to the copper. Examples of anti-infection coatings include drugs, metals, alloys, oxides, nitrides, vitamins, ceramics, plastics, and allotropes of elements such as Carbon (e.g. graphene).

As used herein, the term “three-dimensional” implants and reference to additive and subtractive manufacturing is used to discuss differences in capabilities for lattice and surface finish. It is understood that non-additive manufacturing methods that generate lattice or porous structures typically associated with additively manufactured methods, hybrid methods that use a combination of additive or subtractive methods to generate a final implant design that has a lattice or specific surface finish can also be called a three-dimensional second generation implant.

The present application provides a solution for next generation medical implants—for example spine, interbody fusion, neck, knee, ankle, face, and hip implants—which overcome the shortcomings of existing medical implants, namely the present application provides complex three dimensional structures with improved infection protection. The medical implant solutions provided retain the structural, lattice/porosity benefits of second-generation medical implants, particularly metal based 3D printed titanium implants, while providing exceptional anti-infection properties. This has the potential to significantly improve the quality of life for patients and reduce the mounting costs related to revision surgeries.

One path to reduce infections is by coating medical implants with special materials such as metals, metal coatings have been found to be highly efficacious, or antibiotics. Metal coatings such as silver, copper, and selenium have shown promising results in terms of reduction in infections through decreased bacterial adhesion, enzyme activity inhibition, and production of reactive oxygen species. However, previously known medical implant structures with metal coatings, such as those coated using galvanic deposition, may suffer from both imprecise coatings and thicker coatings such as those having a thickness greater than several micrometers. Additionally, line of sight deposition methods may not optimally cover complex implant structures that by nature have significant geometrical self-shadowing. Thus, using conventional deposition techniques, this shadowing may result in incomplete, non-uniform coatings.

Relating to antibiotic coatings, in limited studies simple techniques such as electrophoresis have been used to coat gentamicin. However, one challenge is that these antibiotic coating processes tend to be thicker, non-selective (i.e., cover the entire implant), and non-conformal. Further, the thicknesses can vary across the complex implant structures due to sharp edges and varying surface areas causing a non-uniform electric field during electrophoresis. For example, in one extreme, on certain parts of the implant the antibiotic may be absent resulting in a fertile ground for infection, while on other parts of the implant the antibiotics may be too thick thus reducing the benefits of the implant's structural intricacies. At very large thicknesses the aforementioned underlying implant properties (such as latticing and porosity induced functions) start to get compromised resulting in possible reduction in osseointegration. Additionally, the range of antibiotics that may be deposited using conventional techniques may be limited.

In another embodiment, ascorbic acid (vitamin C) may be used to enhance the bone formation. This provides an indirect anti-infection activity by accelerating the rate of bone formation and associated vascularization which can enhance the host's ability to defend against infection and decrease the available surface area for pathogenic colonization.

In another embodiment, graphene, an allotrope of carbon is used to alter the ability for pathogens such as bacteria to adhere to an implant and to create a hostile environment for bacterial growth and expansion. Graphene cannot easily be coated directly onto Ti6Al4V, one common alloy used in medical implants. In such an embodiment, a Ti6Al4V or PEEK or PEKK implant would be coated initially with a copper-based layer followed by graphene.

In another embodiment, metals or alloys such as Copper or Ti6Al4V can be coated for anti-microbial activity. Copper has inherent antimicrobial activity while Ti6Al4V can be used to coat stainless steel implants as Ti6Al4V has decreased bacterial adhesion.

In another embodiment, ceramics such as zirconia oxide or alumnia oxide may be used to coat an implant to decrease the surface roughness, thereby decreasing the available surface area for pathogenic colonization and infection. This is particularly useful for areas of an implant that may be in contact with soft tissue and do not require surface roughness.

In another embodiment, oxides and nitrides can be can used as an anti-infection coating. Titanium oxide, Titanium nitride, and Silicon nitride are examples of materials that provide anti-infection through decreased surface adherence of bacteria and increase the rate of bone healing (thereby enhancing host defenses).

The large coating thickness ranges used in existing medical implants, for example coating thicknesses in the range of several micrometers, starts to compromise the aforementioned underlying implant properties of both the medical implant structural material, materials such as titanium and extra low interstitial titanium alloy Ti6Al4V, as well as the those stemming from the highly beneficial lattice structures. Hence, both osseointegration and the ability to negate age-related implant loosening is compromised. In conclusion, current second-generation, state-of-the-art, implants do not effectively prevent infection while maintaining effective osseointegration.

The present application provides medical implants having nano-coatings of materials, such as metals and antibiotics, using precise, low temperature, conformal deposition techniques such as but not limited to atomic layer deposition (ALD), chemical vapor deposition (CVD), ion-based deposition (example: physical vapor deposition), electrochemical deposition (example: electro and electroless plating) processes, and spray-based deposition on the medical implants. These processes are capable of depositing material thicknesses from a few angstroms to nanometer levels and may in some cases exercise angstrom level control of the film thickness. Further, plasma enhanced atomic layer deposition (PEALD) as well as plasma enhanced chemical vapor deposition (PECVD) may be used. Plasma enhanced atomic layer deposition (PEALD) and plasma enhanced chemical vapor deposition (PECVD) have the advantage of further lowering deposition temperature closer to room temperature as compared to their thermal counterparts while maintaining conformal deposition. This is because ion bombardment from plasma aids in providing the necessary activation energy for deposition, reducing the reliance on higher temperatures. Advantageously, lower temperature processes are especially useful for coatings, particularly antibiotics, whose properties are sensitive or may degrade at higher temperatures.

The very thin coatings provided herein, having a material thicknesses from a few angstroms to nanometer levels, are thin enough not to compromise the underlying exceptional intricate structural medical implant properties while being thick enough to only modify implant surfaces to provide exceptional anti-infection efficacy.

The coatings provided to coat implant surfaces have anti-infection properties. These anti-infection metal materials include silver, copper, and selenium, and these anti-infection antibiotics include gentamicin, tobramycin, ciprofloxacin, ampicillin, vancomycin, and rifampin. In a specific embodiment, silver between 1 nm to 50 um may be deposited on spine implants such as cages and screws using chemical vapor deposition at temperatures ranging from 50 C to 1000 C. In another embodiment, gentamicin or tobramycin between 0.5 nm to 1 um may be deposited on spine implants such as cages and screws using atomic layer deposition and plasma enhanced atomic layer deposition at temperatures ranging from 25 C to 100 C. In another embodiment, Ag between 0.5 nm to 500 nm may be deposited on neck, kneed, ankle, face, and hip implants using atomic layer deposition and plasma enhanced atomic layer deposition at temperatures ranging from 25 C to 400 C. These medical implant solutions provide improved anti-infection/antibiotics properties, effective osseointegration, and reduced stress shielding using nanocoating and metal 3D printing.

Challenges relating to coating very thin coatings, for example coating thicknesses less than several micrometers, on complex medical implant structures include that these structures tend to internally shade themselves. In other words, if the deposition technique is only line of sight, geometrical shading will result in uneven coating thicknesses. Hence, some parts of the implant will get the coating, while other parts may get extremely thin or no coating at all.

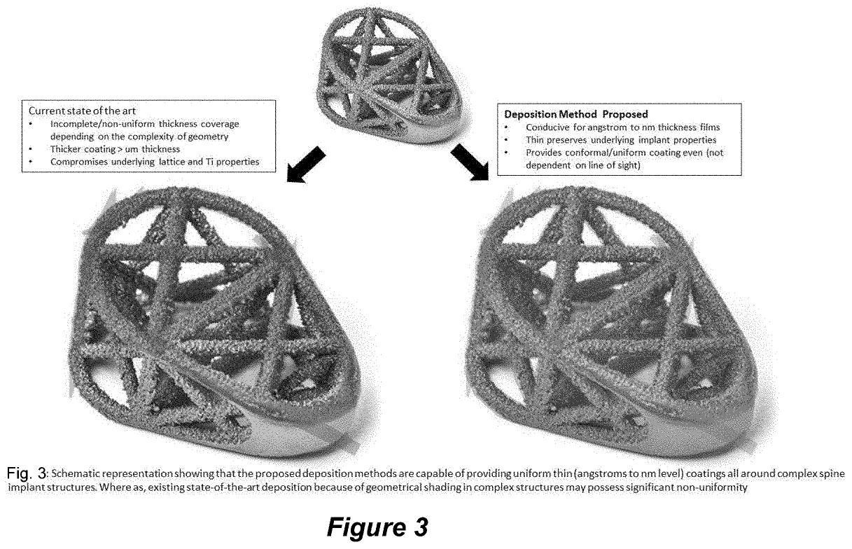

The solutions provided herein, using processes such as atomic layer deposition and chemical vapor deposition which have the advantage of having low surface sticking probability (sticking coefficient) of the gas phase deposited atoms (defined as the probability of a deposited atom sticking on the surface for a given impingement), overcome these geometrical shading challenges. Thus, for low sticking probability methods, the atoms coming on to the substrate tend to bounce around and get in places which otherwise would have been geometrically shielded. This is conceptually shown in FIG. 3. Lower sticking probability methods may result in coverage all around the complex medical implants including in areas which would normally be geometrically shielded from line of sight deposition techniques. The sticking coefficient may be further lowered by modifying the deposition temperature and pressure and by using alternate precursors. Thus, the proposed atomic layer deposition and chemical vapor deposition techniques have the advantage of providing uniform, consistent, and conformal coatings even on complex geometries such as latticed and porous medical implants.

Yet another challenge to coating medical implants stems from their complex three-dimensional geometries and the associated z-heights. Most existing precision deposition processes are conducive for planar semiconductor wafers. A solution provided herein is modifying nanocoating reactors to ensure that the reactors provide the same efficacy and thin film coating advantages on the three-dimensional geometries of medical implants as they do on the conventional wafers. In a specific embodiment, the plasma source is added on the atomic layer deposition reactor to create a plasma enhanced atomic layer deposition (PEALD) designed such that there is enough clearance in the z-direction to accommodate a majority of the medical implant parts.

The foregoing description of the exemplary embodiments is provided to enable any person skilled in the art to make or use the claimed subject matter. Various modifications to these embodiments will be readily apparent to those skilled in the art, and the generic principles defined herein may be applied to other embodiments without the use of the innovative faculty. Thus, the claimed subject matter is not intended to be limited to the embodiments shown herein but is to be accorded the widest scope consistent with the principles and novel features disclosed herein.

What is claimed is:

1. A method for forming a medical implant, the method comprising:
forming a three dimensional structure using three dimensional (3D) printing, said three dimensional structure having x, y, and z structural geometry in a three dimensional x, y, and z Cartesian coordinate system;
coating said three dimensional structure with an anti-infection material having a thickness in the range of 0.5 nanometers to 1.0 micrometers.

2. The method of claim 1, wherein said coating said three dimensional structure with an anti-infection material is performed using atomic layer deposition.

3. The method of claim 2, wherein said coating said three dimensional structure with an anti-infection material is performed using plasma enhanced atomic layer deposition.

4. The method of claim 1, wherein said coating said three dimensional structure with an anti-infection material is performed using chemical vapor deposition.

5. The method of claim 4, wherein said coating said three dimensional structure with an anti-infection material is performed using plasma enhanced chemical vapor deposition.

6. The method of claim 1, wherein said coating is a metal.

7. The method of claim 7, wherein said metal is silver.

8. The method of claim 1, wherein said coating is an antibiotic.

9. The method of claim 8, wherein said antibiotic is gentamicin.

10. The method of claim 3, wherein said coating is a metal.

11. The method of claim 10, wherein said metal is silver.

12. The method of claim 5, wherein said coating is an antibiotic.

13. The method of claim 12, wherein said antibiotic is gentamicin.

14. The method of claim 1, wherein said three dimensional structure is made of titanium.

15. A medical implant, comprising:
a three dimensional structural component having x, y, and z structural geometry in a three dimensional x, y, and z Cartesian coordinate system, said three dimensional structural component made of at least a structural material and a nanocoating;
a nanocoating of anti-infection material having a thickness in the range of 0.5 nanometers to 1.0 micrometers and coating said three dimensional structural material.

16. The medical implant of claim 15, wherein said coating is a metal.

17. The medical implant of claim 16, wherein said metal is silver.

18. The medical implant of claim 17, wherein said coating is an antibiotic.

19. The medical implant of claim 18, wherein said antibiotic is gentamicin.

20. The medical implant of claim 15, wherein said three dimensional structural component is made of titanium.
